(19) United States
(12) Patent Application Publication
(10) Pub. No.: US 2024/0335187 A1
(43) Pub. Date: Oct. 10, 2024

(54) MEDICAL DEVICES AND RELATED METHODS THEREOF

(71) Applicant: Boston Scientific Scimed, Inc., Maple Grove, MN (US)

(72) Inventors: Michael JACOBS, Franconia, NH (US); Bryan BANNON, Duxbury, MA (US); Prateek MAHESHWARI, Boston, MA (US); James WELDON, Newton, MA (US)

(73) Assignee: Boston Scientific Scimed, Inc., Maple Grove, MN (US)

(21) Appl. No.: 18/627,881

(22) Filed: Apr. 5, 2024

Related U.S. Application Data
(60) Provisional application No. 63/494,960, filed on Apr. 7, 2023.

(51) Int. Cl. A61B 10/04; A61B 1/00; A61B 1/018; A61B 10/02

(52) U.S. Cl. CPC A61B 10/04; A61B 1/00098; A61B 1/018; A61B 2010/0216

(57) ABSTRACT

A medical device including an expandable tool configured to extend from a distal end of the medical device. The expandable tool includes a plurality of wires extending from a proximal end of the expandable tool to a distal end of the expandable tool, a plurality of fins extending outwardly from each of the wires of the plurality of wires, and a plurality of projections extending outwardly from each of the fins of the plurality of fins.

### CROSS-REFERENCE TO RELATED APPLICATIONS

This application claims the benefit of priority under 35 U.S.C. § 119 from U.S. Provisional Application No. 63/494,960, filed Apr. 7, 2023, which is incorporated by reference herein in its entirety.

### TECHNICAL FIELD

Various aspects of this disclosure relate generally to medical systems, devices, and related methods thereof. More specifically, embodiments of the disclosure relate to devices for collecting tissue samples, for example, tissue samples within the common bile duct and the pancreatic duct.

### BACKGROUND

Endoscopic retrograde cholangiopancreatography (ERCP) is a procedure that utilizes endoscopy and fluoroscopy to diagnose and/or treat conditions of the biliary and pancreatic ductal systems, such as strictures. During an exemplary ERCP procedure, an endoscope may be inserted into a patient's mouth and navigated down the esophagus through the stomach into the duodenum and to the papilla, where each of the common bile duct (of the biliary ductal system) and the pancreatic duct enter the duodenum. A contrast agent may then be injected, so that an operator may use fluoroscopy and visualize a stricture within the biliary or pancreatic ductal systems on an x-ray image. The operator may then use an instrument, for example a cytology brush, to collect a sample of tissue from the stricture to help diagnose malignant stenosis.

Cytology brushes used during an ERCP procedures often have diameters that are too large for use with endoscopes including imaging devices, as the working channels of such endoscopes typically have reduced diameters in order to accommodate the imaging devices. Therefore, cytology brushing conducted during an ERCP procedure is often conducted blindly once the operator has positioned the endoscope in the general vicinity of the stricture. As a result, it may be difficult for the operator to accurately collect an adequate tissue sample from the stricture, as the brush may inadvertently be inserted into the wrong duct (e.g., into the biliary duct and not into the pancreatic duct), or may not come into contact with the stricture despite repeated probing. Such difficultly caused by the lack of visualization may prolong a length of the procedure, or may result in collection of an inadequate tissue sample. Other procedures also may involve biopsies using cytology brushes and may encounter similar difficulties as those described above. Therefore, a need exists for devices for collecting tissue samples.

### SUMMARY

Each of the aspects disclosed herein may include one or more of the features described in connection with any of the other disclosed aspects.

Aspects of the disclosure relate to, among other things, systems, devices, and methods for collecting tissue samples, for example, tissue samples within the common bile duct and the pancreatic duct, during medical procedures. Aspects of the disclosure also relate to, among other things, expandable tools having features, such as bristles or wires for collecting increased volumes of tissue.

According to an example, a medical device may include an expandable tool configured to extend from a distal end of the medical device, the expandable tool including a plurality of wires extending from a proximal end of the expandable tool to a distal end of the expandable tool, a plurality of fins extending outwardly from each of the wires of the plurality of wires, and a plurality of projections extending outwardly from each of the fins of the plurality of fins.

Any of the medical devices described herein may include any of the following features. The plurality of wires defines a plurality of legs, where each of the legs of the plurality of legs branch out from a central trunk disposed at the proximal end of the expandable tool. The plurality of legs are joined together at the distal end of the expandable tool. The expandable tool is configured to be transitioned from a collapsed configuration to an expanded configuration. In the collapsed configuration, each of the fins of the plurality of fins is configured to collapse inward toward the plurality of wires. In the expanded configuration, each of the wires of the plurality of wires is configured to bow outward such that each of the wires has a concave profile. In the collapsed configuration, an outer diameter of the expandable tool is no greater than 1.5 mm. In the expanded configuration, a width of the expandable tool is in a range of 1 mm to 15 mm. The wires of the plurality of wires include a first material, and the fins of the plurality of fins include a second material. The first material is the same as the second material. The projections of the plurality of projections comprise a third material. The third material is the same as the second material. The fins of the plurality of fins are integrally formed with the wires of the plurality of wires. The projections of the plurality of projections extend radially outward relative to a longitudinal axis of each of the fins of the plurality of fins. The wires of the plurality of wires extend approximately parallel to a longitudinal axis of the expandable tool in a collapsed configuration of the expandable tool.

According to another example, a medical device may include an expandable tool configured to extend from a distal end of the medical device, the expandable tool including a plurality of wires extending from a proximal end of the expandable tool to a distal end of the expandable tool. In an expanded configuration of the expandable tool, each of the wires of the plurality of wires may be configured to be curved outward to form a basket, each of the wires of the plurality of wires may include a first portion configured to extend inwardly relative to a profile of the wire, and a second portion configured to extend outwardly relative to the profile of the wire, and the wires of the plurality of wires may be configured to be joined together at the distal end of the expandable tool.

Any of the medical devices described herein may include any of the following features. The wires of the plurality of wires include sharpened wires. The wires of the plurality of wires include textured wires. Each of the textured wires includes a plurality of projections extending outward and away from a longitudinal axis of each of the textured wires. Each of the projections of the plurality of projections includes a free end configured to form a barb extending radially away from the longitudinal axis of each of the textured wires. Each of the wires of the plurality of wires includes a shape memory material. In a collapsed configuration of the expandable tool, the profile of the wire extends approximately parallel to a longitudinal axis of the expandable tool.

According to another example, a method for collecting a tissue sample using a medical device may include navigating an expandable tool to a target site within a lumen of a patient. The expandable tool may include a plurality of wires extending from a proximal end of the expandable tool to a distal end of the expandable tool, a plurality of fins extending outwardly from each of the wires of the plurality of wires, and a plurality of projections extending outwardly from each of the fins of the plurality of fins. The method may include transitioning the expandable tool from a collapsed configuration to an expanded configuration and contacting a wall of the lumen with the plurality of projections such that the tissue sample is collected on the projections.

Any of the methods described herein may include any of the following features. After contacting the wall of the lumen, transitioning the expandable tool from the expanded configuration to the collapsed configuration. Transitioning the expandable tool from the collapsed configuration to the expanded configuration comprises actuating an actuation wire coupled to the expandable tool.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “distal” refers to a direction away from an operator/toward a treatment site, and the term “proximal” refers to a direction toward an operator. The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows a perspective view of an exemplary medical device according to some embodiments.

FIG. 2A shows a side view of an exemplary cytology brush in a collapsed configuration according to some embodiments.

FIG. 2B shows a side view of the cytology brush of FIG. 2A in an expanded configuration according to some embodiments.

FIG. 3A shows a side view of an exemplary cytology basket in a collapsed configuration according to some embodiments.

FIG. 3B shows a side view of the cytology basket of FIG. 3A in an expanded configuration according to some embodiments.

FIG. 4 shows top and side views of a wire used in an exemplary cytology basket according to some embodiments.

FIG. 5 shows top and side views of another wire used in an exemplary cytology basket according to some embodiments.

FIG. 6A shows a side view of an exemplary cytology snare in a collapsed configuration according to some embodiments.

FIG. 6B shows a side view of the cytology snare of FIG. 6A in an expanded configuration according to some embodiments.

### DETAILED DESCRIPTION

Embodiments of this disclosure relate to medical devices including structures configured to collect tissue samples from lumen within the body of a patient (for example, within the common bile duct and/or the pancreatic duct) during a medical procedure, for example during an endoscopic retrograde cholangiopancreatography (ERCP) procedure. During an ERCP procedure, a physician, or other operator, may utilize both endoscopic and fluoroscopic techniques to diagnose and treat issues arising in the common bile duct and in the pancreatic duct. In some instances, these issues may include the presence of strictures, which may need to be analyzed to determine whether they are malignant. To analyze a stricture, a sample of tissue may need to be collected from the affected area. Although ERCP procedures are referenced herein, it will be appreciated that the disclosed devices and methods may be utilized in the course of other medical procedures (for example, during medical procedures that involve obtaining biopsy samples of lesions).

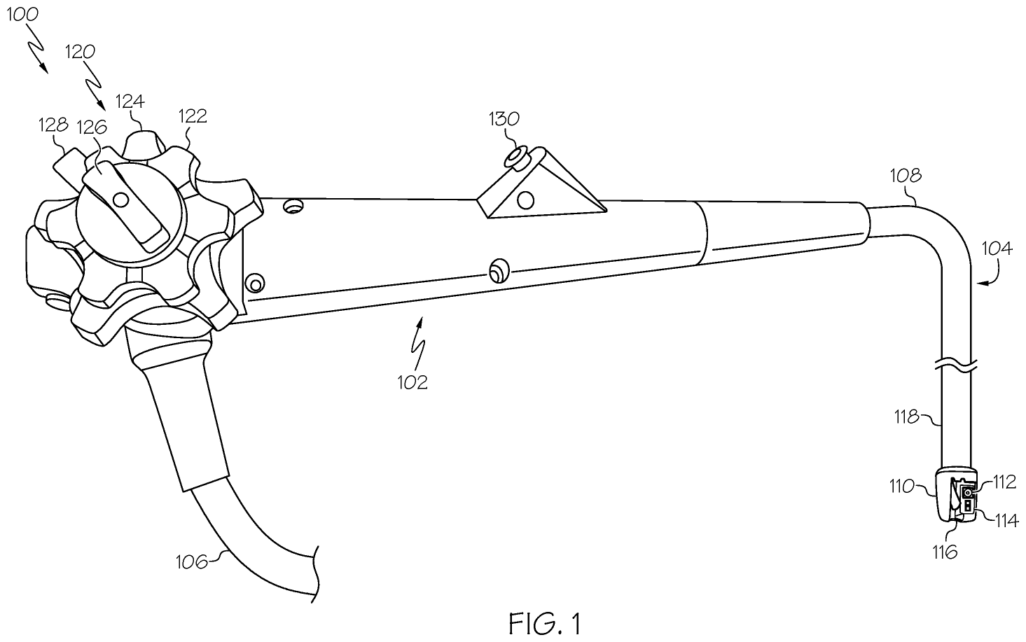

FIG. 1 depicts an exemplary medical device 100 according to some embodiments. In some examples, medical device 100 may be a scope (such as a duodenoscope, endoscope, cholangioscopes, ureteroscope, bronchoscope, cystoscope, colonoscope, laparoscope, or laryngoscope), tome, catheter, or sheath. Medical device 100 may include a handle 102 and an insertion portion 104. Medical device 100 may also include an umbilicus 106 for purposes of connecting medical device 100 to sources of, for example, air, water, suction, power, etc., as well as to image processing and/or viewing equipment.

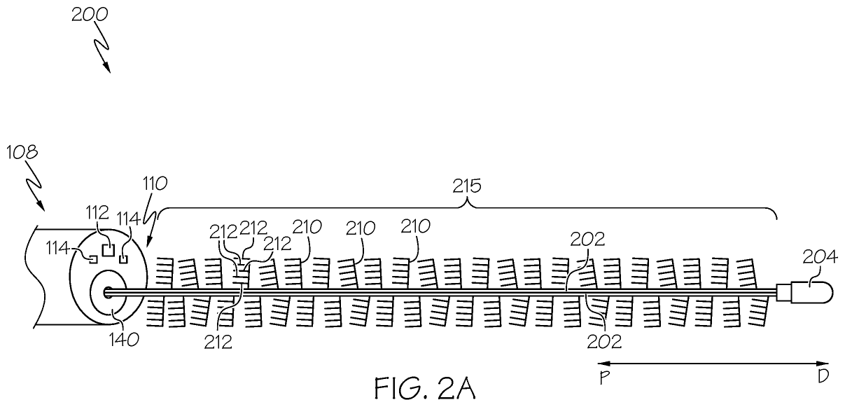

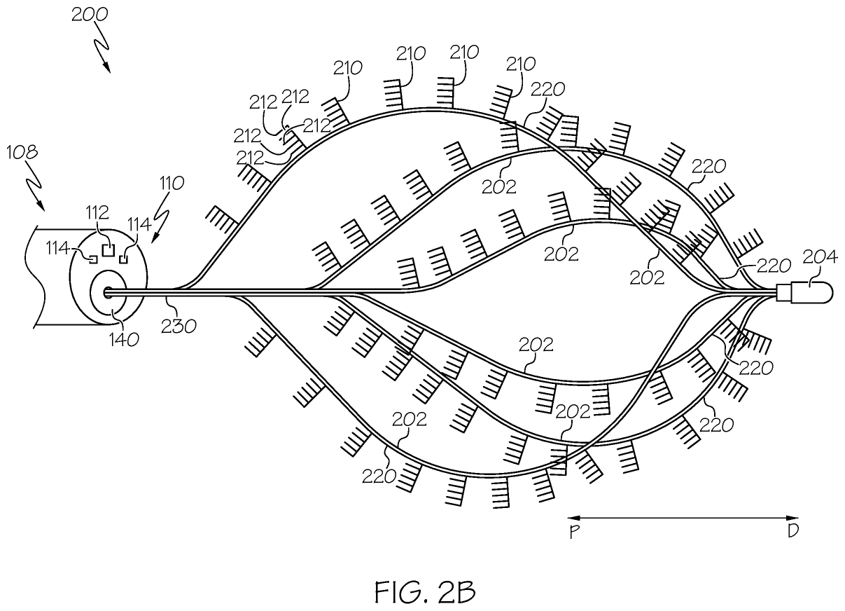

Insertion portion 104 may include a sheath or shaft 108 and a distal tip 110. In some embodiments, distal tip 110 may include one or more imaging devices 112 (e.g., one or more cameras) for capturing images, and may also include one or more illumination devices 114 (e.g., one or more light emitting diodes (LEDs) or optical fibers) for providing illumination to facilitate image capture and visualization. Distal tip 110 may be side-facing such that imaging device 112 and illumination devices 114 may face radially outward, perpendicularly, approximately perpendicularly, or otherwise transverse to a longitudinal axis of shaft 108 and distal tip 110. However, distal tip 110 may alternatively be “forward facing” (i.e., distal-facing), for example as shown in FIGS. 2, 3, and 6. Although one imaging device 112 and two illumination devices 114 are depicted in FIG. 1, any suitable number of imaging devices 112 and/or illumination devices 114 may be utilized. Alternatively, imaging device 112 and illumination devices 114 may be combined into a single device.

In some embodiments, an elevator 116 may be disposed at distal tip 110. Elevator 116 may be configured to change an orientation of a tool, for example a cytology tool, inserted in a working channel (e.g., a working channel 140, shown in FIGS. 2, 3, and 6) of medical device 100. Elevator 116 may alternatively be referred to as a swing stand, pivot stand, raising base, or any suitable other term. In some embodiments, elevator 116 may be pivotable via, e.g., an actuation wire or another control element that extends from handle 102, through shaft 108, to elevator 116.

Distal tip 110 may also include components in addition to or in the alternative to the components described above. For example, distal tip 110 may include additional or alternative sources of lighting and/or additional or alternative imaging components (e.g., additional cameras). Distal tip 110 may also include additional types of sensors, such as moisture sensors, temperature sensors, pressure sensors, or other types of sensors, which may be useful during a medical procedure.

In some embodiments, a distal portion of shaft 108 that is connected to distal tip 110 may have a steerable section 118. Steerable section 118 may be, for example, an articulation joint. Shaft 108 and steerable section 118 may include a variety of structures, which are known or may become known in the art.

Handle 102 may have one or more actuators/control mechanisms 120. Control mechanisms 120 may provide control over steerable section 118 or may allow for provision of air, water, suction, etc. For example, handle 102 may include control knobs 122, 124 for left, right, up, and/or down control of steerable section 118. For example, one of knobs 122, 124 may provide left/right control of steerable section 118, and the other of knobs 122, 124 may provide up/down control of steerable section 118. Handle 102 may further include one or more locking mechanisms 126 (e.g., knobs or levers) for preventing steering of steerable section 118 in at least one of an up, down, left, or right direction. In some embodiments, handle 102 may also include an elevator control lever 128. Elevator control lever 128 may raise and/or lower elevator 116, via connection between lever 128 and an actuating wire (not shown) that extends from lever 128, through shaft 108, to elevator 116. A port 130 may allow passage of a tool through port 130, into a working channel (e.g., working channel 140, shown in FIGS. 2, 3, and 6) of the medical device 100, through shaft 108, to distal tip 110. Although not shown, handle 102 may also include one or more valves, buttons, actuators, etc. to control the provision of air, water, suction, etc.

In use, an operator may insert at least a portion of shaft 108 into a body lumen of a patient, and distal tip 110 may be navigated to a target site in the body lumen. For an ERCP procedure, distal tip 110 may be inserted into a patient's mouth and navigated down the esophagus through the stomach, into the duodenum, and to the location of a stricture. The operator may then insert a tool into port 130, and pass the tool through shaft 108 via the working channel to distal tip 110. The tool may exit the working channel via an opening at distal tip 110. The operator may use elevator control lever 128 to raise elevator 116 and angle the tool toward the location of the stricture.

Conventional cytology tools, however, typically have diameters or widths that are too large to fit through the working channel of such medical devices having imaging and illumination devices disposed at their distal tips, as the working channel in these devices is often smaller than those within endoscopes or cholangioscopes not having these features. A smaller, conventional cytology tool that would fit through the working channel of such medical device having illumination and imaging devices may fail to capture an adequate sample (e.g., may capture too few cells, may not exert enough pressure on the sampled tissue (e.g., stricture), or may otherwise be inadequate. Accordingly, cytology tools are typically used with endoscopes and cholangioscopes that do not include built-in imaging devices. As a result, when a cytology tool is used to collect a tissue sample, it is often conducted blindly, as an imaging device may need to be removed from the working channel before the cytology tool can be inserted.

Collecting tissue samples blindly may have limitations. For example, because the operator cannot see precisely where the cytology tool is being placed, it may be more difficult for the operator to accurately collect a tissue sample from the stricture, and it may also be more likely for the operator to misplace the tool, which could lead to the tool being inadvertently advanced into the pancreatic duct.

The cytology tools described herein may be collapsible, and may be sized to fit through the working channel of a medical device having a distal tip including imaging and illumination devices, such as medical device 100. The cytology tools described herein also may facilitate visualization of the sample collection because the cytology tools may not block a view of the imaging device.

For example, FIGS. 2A and 2B show side views of an exemplary cytology brush, according to some embodiments. In some embodiments, a cytology brush 200 may be configured to be transitioned from a collapsed configuration, as shown in FIG. 2A, to an expanded configuration, as shown in FIG. 2B. While in the collapsed configuration, cytology brush 200 may have an outer diameter small enough to fit through a working channel of a medical device having imaging and illumination devices disposed at its distal tip. For example, the outer diameter of cytology brush 200 may be small enough to fit through working channel 140 of medical device 100, described above. In some embodiments, a maximum outer diameter of cytology brush 200 may be in a range of 1 mm to 2 mm, including subranges, when it is in the collapsed configuration. In some embodiments, the maximum outer diameter of cytology brush 200 may be up to approximately 1.5 mm when it is in the collapsed configuration. Accordingly, cytology brush 200 may be used in conjunction with an imaging device of medical device 100, for example imaging device 112, which may allow an operator to more accurately position cytology brush 200 adjacent a stricture, as direct visualization of the stricture during placement of cytology brush 200 is possible.

Once cytology brush 200 is positioned in a desired location, it may be transitioned to an expanded configuration, for example as shown in FIG. 2B. In the expanded configuration, a width of cytology brush 200 (in a direction perpendicular to a longitudinal axis of medical device 100/cytology brush 200) may be in a range of approximately 1 mm to up to approximately 15 mm, including subranges (e.g., a range of approximately 1.0 mm to approximately 10.0 mm), which may be significantly larger than a width of a typical cytology brush. Accordingly, cytology brush 200 may contact a stricture more easily, as it can expand within a body lumen until it contacts the stricture. By making direct contact with the stricture upon expansion, repetitive advancement and retraction of cytology brush 200 against the stricture may be largely avoided, thereby helping to minimize trauma. Furthermore, a larger surface area of cytology brush 200 may allow for collection of an adequate (i.e., large enough) number of sample cells without repeated advancement and retraction of cytology brush 200.

As shown in FIG. 2B, cytology brush 200 may be in the form of a basket having a plurality of wires 202 extending from a proximal end of cytology brush 200 to a distal end of cytology brush 200, which may be configured to form a basket shape. In some embodiments, cytology brush may include up to 8 wires, but any number of wires suitable to form a basket may be used. Wires 202 may be formed from a shape memory material, for example nitinol, but any material suitable for use in cytology tools may be used. Wires 202 may define a plurality of legs 220, which may branch out from one or more central trunk(s) 230. The legs 220 may join together at a distal tip 204. Legs 220 may branch from trunk 230 at a same location or, as shown in FIG. 2B, legs 220 may branch from trunk 230 at locations that are staggered proximally/distally along trunk 230. Thus, legs 220 may be joined together at their proximal and distal ends and may branch outward from one another at locations between the proximal and distal ends. In the collapsed configuration, wires 202 may extend approximately parallel to a longitudinal axis of cytology brush 200. In the expanded configuration, wires 202 may bow outward, such that wires 202 have a concave shape.

In some embodiments, a plurality of fins 210 may extend outwardly from each of wires 202 (i.e., from each of the legs 220), and fins 210 may each include a plurality of outward-extending projections 212. Fins 210 may be configured to extend outwardly from each of wires 202 when cytology brush 200 is in the expanded configuration, but may optionally be collapsible when cytology brush 200 is in the collapsed configuration such that they may fold inward against wires 202 while cytology brush 200 is being advanced through the working channel of a medical device. The number of fins 210 on each of wires 202 may vary, and any number of fins 210 may be incorporated onto each of wires 202 as is feasible.

In some embodiments, projections 212 may be bristles, for example bristles typically used in a cytology brush, and may be made from a polymeric material stiff enough to dislodge cells from a stricture, but soft enough to avoid damaging a duct wall. For example, in some embodiments, the bristles may be made from a material such as nylon, polypropylene, polyimide, or polyether ether ketone (PEEK). Additionally, the bristles may optionally include braided or wound filaments. Fins 210 may be similar to smaller versions of traditional cytology brushes formed on wires 202. Fins 210 may be integrally (monolithically) formed with wires 202; however, fins 210 may alternatively be separate elements that are secured to wires 202. Wires 202 and fins 210 may be constructed from the same material or from different materials. Projections 212 may be formed integrally (monolithically) with fins 210, or projections 212 may be separately formed from fins 210 and attached to fins 210. Projections 212 and fins 210 may be formed of the same materials as fins 210 or from different materials. In some examples, projections 212 may be formed from any material that bristles of a cytology brush would be formed from.

As shown in FIG. 2B, projections 212 may extend in only one direction from fins 210. For example, as shown in FIG. 2B, fins 210 may extend approximately perpendicularly from wires 202 in an expanded configuration of cytology brush 200, and projections 212 may extend from sides of fins 210 that face proximally. Projections 212 may extend approximately parallel to wires 202. Where wires 202 extend approximately parallel to a longitudinal axis of cytology brush 200 (i.e., approximately along an axis that extends proximally/distally), projections 212 may extend approximately proximally (i.e., approximately parallel to wires 202, extending in a proximal direction). Projections 212 may be configured to gather a sample as cytology brush 200 is moved in a proximal direction. In addition or in the alternative, projections 212 may extend in other directions (distally or perpendicularly to a longitudinal axis of cytology brush 200). In some examples, projections 212 may extend radially outward relative to a longitudinal axis of each of fins 210. Each of fins 210 may include one or more rows of projections 212.

The incorporation of fins 210 onto wires 202 may increase the surface area of cytology brush 200, as tissue may be collected on the surfaces of each of projections 212 or on a subset of projections 212 that face/contact a stricture or other structure being sampled This increased surface area may aid in collection of larger tissue samples than a typical cytology brush, which may be helpful in analyzing whether the stricture is malignant. A sample may be collected on projections 212 from a plurality of fins 210 and may be captured on projections 212 of a plurality of fins 210 disposed on a plurality of different wires 202.

Additionally, in some embodiments, a length of cytology brush 200 may be longer than that of a typical cytology brush. The increased length of cytology brush 200 may allow for cytology brush 200 to span the length of a working channel of a medical device, and extend beyond, but remain within the field of view of, an imaging device, for example imaging device 112. In some embodiments the length of cytology brush 200 is in a range of 230 cm to 240 cm, including subranges. Additionally, a length 215 of the portion of cytology brush 200 including fins 210 may be in a range of 5 mm to 15 mm, including subranges.

In some embodiments, distal tip 204 of cytology brush 200 may include an atraumatic distal tip 204 disposed at a distalmost end of cytology brush 200. As discussed above, distal tip 204 may connect wires 202 together, and may provide a blunt surface at the distal end of cytology brush 200, which may aid in preventing undesired trauma to tissue during an ERCP procedure.

To collect a tissue sample from a stricture during an ERCP procedure, for example using medical device 100, shaft 108 may inserted into a body lumen of a patient, and distal tip 110 may be navigated to a target site, for example to a location of a stricture, with the aid of imaging device 112 and illumination device(s) 114. Once distal tip 110 is adjacent the stricture, cytology brush 200, while in the collapsed configuration, may be inserted into port 130. Cytology brush 200 may then pass through shaft 108 via working channel 140 to distal tip 110, where it may exit shaft 108. The operator may then utilize imaging device 112 to help advance cytology brush 200 to a position adjacent the stricture before transitioning cytology brush 200 from the collapsed configuration to the expanded configuration. In some embodiments, elevator 116 may be utilized to aid in the positioning of cytology brush 200.

To transition cytology brush 200 from the collapsed configuration to the expanded configuration, the operator may engage an actuation wire (not shown), which may be coupled to cytology brush 200. For example, the operator may pull the actuation wire in a proximal direction, which may cause cytology brush 200 to expand. Once cytology brush 200 is transitioned to the expanded configuration, the operator may contact a wall of the lumen at the location of the stricture with fins 210 such that bristles 212 may collect samples of tissue from the stricture. For example, in some examples, legs 220 may be moved proximally relative to trunk 230. In addition or in the alternative, trunk 230 may be moved distally relative to legs 220. Additionally or alternatively, wires 202 may have shape memory properties (e.g., may be biased into an expanded configuration).

After a tissue sample has been collected, cytology brush 200 may be transitioned back to the collapsed configuration so it may be removed from medical device 100 via shaft 108, and through port 130. For example, legs 220 may be moved distally relative to trunk 230 and/or trunk 230 may be moved proximally relative to legs 220. Additionally or alternatively, a sheath may be deployed to constrain wires 202.

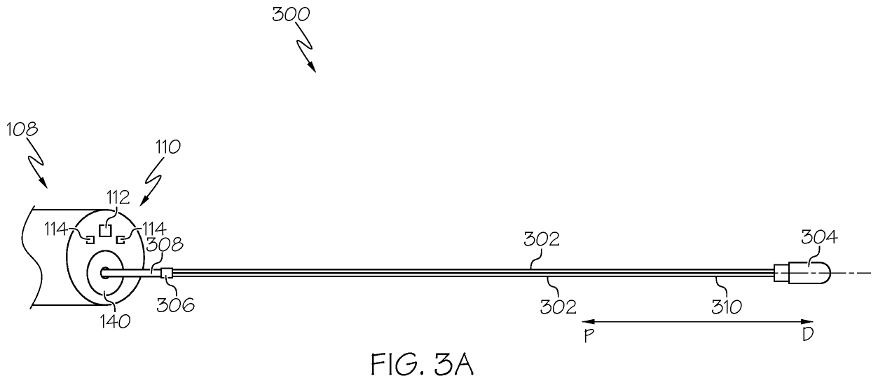

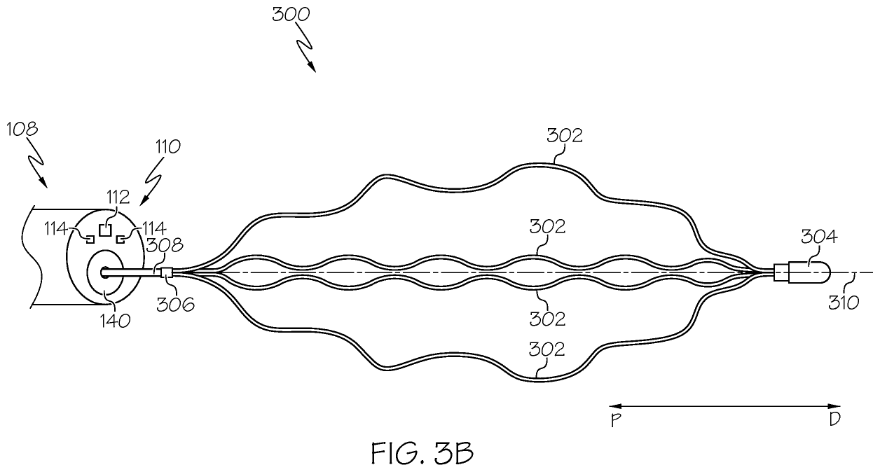

The cytology tools described herein may also include a cytology basket. For example, FIGS. 3A and 3B show side views of an exemplary cytology basket according to some embodiments.

Similar to cytology brush 200, described above, cytology basket 300 may be configured to be transitioned from a collapsed configuration, as shown in FIG. 3A, to an expanded configuration, as shown in FIG. 3B. Unless specified herein, cytology basket 300 may have any of the same features of cytology brush 200. While in the collapsed configuration, cytology basket 300 may have an outer diameter small enough to fit through a working channel of a medical device having imaging and illumination devices disposed at its distal tip. For example, the outer diameter of cytology basket 300 may be small enough to fit through working channel 140 of medical device 100, described above. Accordingly, cytology basket 300 may be used in conjunction with an imaging device of medical device 100, for example imaging device 112.

Cytology basket 300 may include a plurality of wires 302 extending from a proximal end of cytology basket 300 to a distal end of cytology basket 300, and, in the expanded configuration, may be curved outward, with respect to a central longitudinal axis 310 of cytology basket 300, to form a basket shape. Although cytology basket 300 is shown having four wires, any number of wires may be used. As shown in FIG. 3B, wires 302 may include portions that extend/protrude/project inwardly/outwardly relative to an overall shape of wires 302. Each of wires 302 may define a longitudinal axis, and projections may extend away from the respective longitudinal axis in any direction (e.g., including a direction extending radially away from longitudinal axis 310). For example, as shown, wires 302 may have an irregular, undulating, or wavy profile. Wires 302 may have such an irregular profile (e.g., having projections/a wavy profile) in the collapsed configuration, as well as in the expanded configuration. The profile may have any desired shape with any number of projections/undulations.

A shape of wires 302 may aid in dislodging tissue from a stricture, as the irregular surface may provide an increased surface area, and may allow for increased agitation of the tissue. Projections/undulations of wires 302 (e.g., projections/undulations that extend away from longitudinal axis 310) may engage tissue to collect sample(s). Alternatively, in some embodiments, wires 302 may have a regular, profile. For example, wires 302 may have a straight configuration in a collapsed configuration of cytology basket 300. In other words, each wire 302 may define a longitudinal axis, and wire 302 may extend along the respective longitudinal axis without projections/undulations. In some embodiments, wires 302 may be formed of a shape memory material, for example nitinol, but any material suitable for use in cytology tools may be used.

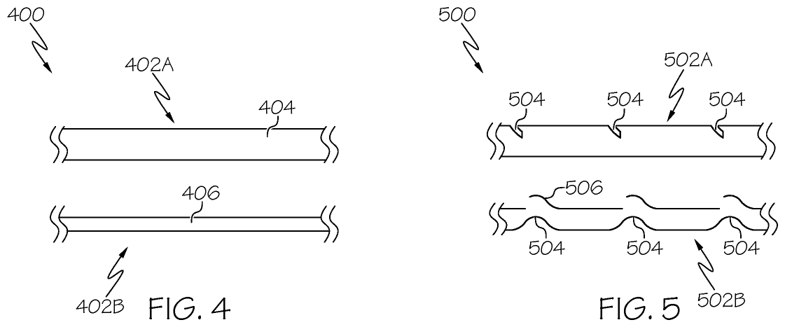

In some embodiments, cytology basket 300 may additionally, or alternatively, include wires 302 having different textures, each configured to improve collection of tissue. For example, FIG. 4 shows top and side views of a sharpened wire 400, while FIG. 5 shows top and side views of a textured wire 500. Some or all of wires 302 of cytology basket 300 may include the features shown in FIGS. 4 and 5. Alternatively, the wires 302 may lack the features shown in FIGS. 4 and 5.

As shown in top and side views 402A and 402B, respectively, of sharpened wire 400, sharpened wire 400 may have a top face 404 and a side face 406. In some embodiments, top face 404 may be substantially flat, while side face 406 may include a sharpened edge configured to cut tissue. Side face 406 may be oriented on cytology basket 300 such that side face 406 faces a tissue (e.g., a stricture) to be sampled. For example, side face 406 may be oriented so as to face approximately radially outward relative to longitudinal axis 310. In some examples, a location of side face 406 may change over a length of sharpened wire 402, such that different portions of wires 302 of basket 300 have a sharpened side face 406 facing in different directions. Different wires 302 of basket 300 may include features of wire 400 having side face 406 facing in different directions.

As shown in top and side views 502A and 502B, respectively, of textured wire 500, textured wire 500 may include a plurality of notches 504. In some embodiments, notches 504 may be configured to snag, or cut tissue. Notches 504 may also be configured to collect small samples of tissue therein. As shown in view 502B, textured wire 500 also may include projections 506 that extend outward, away from a longitudinal axis of textured wire 500. Projections 506 may include a free end that forms a barb extending radially away from a longitudinal axis of textured wire 500. A wire (such as wire 302) may include a portion that is textured as textured wire 500 and a portion that is smooth or has the sharpened edge of wire 400. The features of wires 400, 500 may be used in any combination on the same wire or on different wires of the same device and/or may be combined with smooth portions on the same wire and/or on different wires of the same device.

Sharpened wire 400 and textured wire 500 may be formed with either a wavy profile or a straight profile. As discussed above, wires 302 of cytology basket 300 may incorporate any of the features of wires 400, 500. Wires 202 of cytology brush 200 also may include features of wires 400, 500.

In some embodiments, cytology basket 300 may also include an atraumatic tip 304 disposed at a distalmost end of cytology basket 300. Atraumatic tip 304 may connect wires 302 together, and may provide a blunt surface at the distal end of cytology basket 300, which may aid in preventing undesired trauma to tissue during an ERCP procedure. Additionally, cytology basket 300 may include a joint 306 disposed at its proximal end, which may be configured to connect cytology basket 300 to an actuation wire 308 or a sheath.

In some embodiments, cytology basket 300 may be utilized during an ERCP procedure in a similar manner to cytology brush 200, described above. For example, once shaft 108 is inserted into a body lumen of a patient, and distal tip 110 has been navigated to a target site, for example a location of a stricture, cytology basket 300, while in the collapsed configuration, may be inserted into port 130. Cytology basket 300 may then pass through shaft 108 via working channel 140 to distal tip 110, where it may exit shaft 108. The operator may then utilize imaging device 112 to help advance cytology basket 300 to a position adjacent the stricture before transitioning cytology basket 300 from the collapsed configuration to the expanded configuration. In some embodiments, elevator 116 may be utilized to aid in the positioning of cytology basket 300.

To transition cytology basket 300 from the collapsed configuration to the expanded configuration, the operator may engage actuation wire 308. For example, the operator may pull actuation wire 308 in a proximal (or distal) direction, which may cause cytology basket 300 to expand. Other, alternative methods may be used to transition cytology basket 300, including any of those methods disclosed above with respect to cytology brush 200. Once cytology basket 300 is transitioned to the expanded configuration, the operator may contact the stricture or other tissue with wires 302, thereby allowing wires 302 to scrape or cut the tissue of the stricture or other tissue. In some embodiments, cytology basket may be twisted against the tissue such that the edges of wires 302 may scrape against the stricture. Alternatively, cytology basket 300 may be horizontally actuated. In other words, once cytology basket 300 is transitioned to the expanded configuration, the operator may position wires 302 against the tissue of the stricture, and then advance and retract cytology basket 300 such that wires 302 scrape against the tissue multiple times. Once tissue within the stricture has been dislodged by wires 302, a suction feature of medical device 100 may be utilize to suck the dislodged tissue through working channel 140 and into a biopsy trap.

After the tissue sample has been collected, cytology basket 300 may be transitioned back to the collapsed configuration (e.g., by moving actuation wire 308 in an opposite direction to the direction actuation wire 308 is moved to expand cytology basket 300) so it may be removed from medical device 100 via shaft 108, and through port 130. Alternatively or additionally, any of the methods described above for cytology basket 300 may be utilized in order to collapse cytology basket 300.

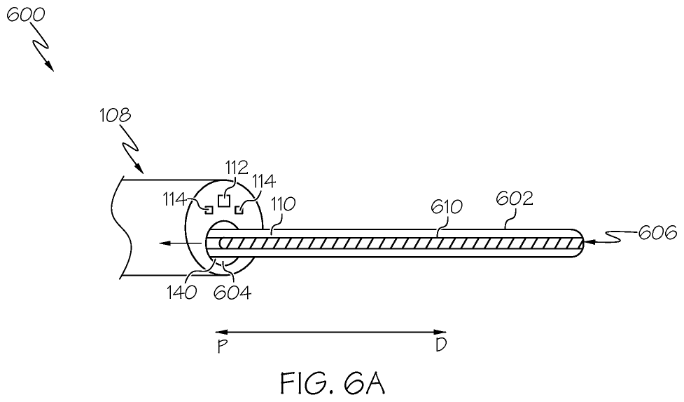

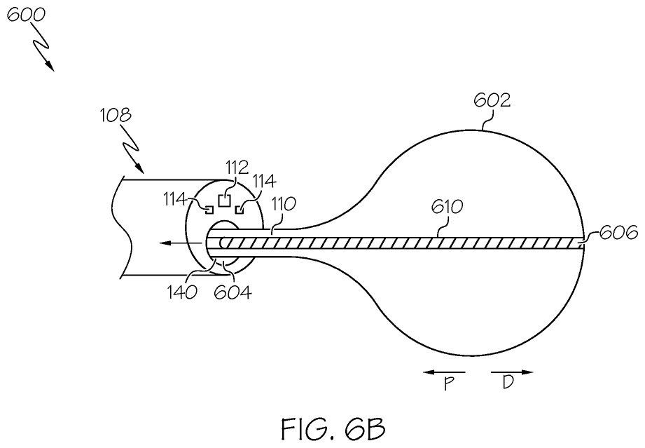

Alternatively, some embodiments of the cytology tools described herein may include a cytology snare. For example, FIGS. 6A and 6B show side views of an exemplary cytology snare 600 according to some embodiments.

Similar to cytology brush 200 and cytology basket 300, described above, cytology snare 600 may also be configured to be transitioned from a collapsed configuration, as shown in FIG. 6A, to an expanded configuration, as shown in FIG. 6B. While in the collapsed configuration, cytology snare 600 may have an outer diameter small enough to fit through a working channel of a medical device having imaging and illumination devices disposed at its distal tip. For example, the outer diameter of cytology snare 600 may be small enough to fit through working channel 140 of medical device 100, described above. Accordingly, cytology snare 600 may be used in conjunction with an imaging device, for example imaging device 112 of medical device 100.

Cytology snare 600 may include a snare wire 602 in the form of a loop extending from a proximal end 604 of cytology snare 600 to a distal end 606 of cytology snare 600. Snare wire 602 may function similarly to the wires 302 of cytology basket 300, discussed above. For example, snare wire 602 may be utilized to scrape or cut tissue within a stricture or other anatomical structure. In some embodiments, snare wire 602 may be formed from a shape memory material, for example nitinol, but any material suitable for use in cytology tools may be used.

Cytology snare 600 may additionally include an actuation wire 610 extending from proximal end 604 to distal end 606, and coupled to snare wire 602 at distal end 606. During use, actuation wire 610 may be pulled proximally with respect to a medical device, for example medical device 100, discussed above, to transition cytology snare 600 from the collapsed configuration to the expanded configuration. For example, pulling actuation wire 610 proximally may cause actuation wire 610 to pull distal end 606 of snare 600 proximally, thereby causing snare wire 602 to expand into a loop, for example as shown in FIG. 6B. The tension caused by pulling actuation wire 610 proximally may also provide snare wire 602 with added rigidity, which may aid in the process of dislodging tissue from a stricture. Furthermore, the amount of tension applied to actuation wire 610 may function to adjust a diameter of cytology snare 600. For example, a greater amount of applied tension to actuation wire 610 may result in a larger diameter of cytology snare 600, while a smaller amount of tension applied to actuation wire 610 may result in a smaller diameter of cytology snare 600. Accordingly, cytology snare 600 may be adjusted to fit within body lumen having varying diameters. Snare wire 602 may have any of the properties of wires 302, 400, 500 discussed above (e.g., undulating shape, sharpened portions, and/or textured portions).

In some embodiments, cytology snare 600 may include a breakaway feature, for example a clip, disposed at proximal end 604. The breakaway feature may allow for cytology snare 600 to easily be released from a wire or a catheter after tissue collection is completed. Cytology brush 200 and/or cytology basket 300 also may include such a breakaway feature.

During an ERCP procedure, cytology snare 600 may be utilized in a similar manner to cytology brush 200 and cytology basket 300, described above. For example, once shaft 108 is inserted into a body lumen of a patient, and distal tip 110 has been navigated to a target site, for example a location of a stricture, cytology snare 600, while in the collapsed configuration, may be inserted into port 130. Cytology snare 600 may then pass through shaft 108 via working channel 140 to distal tip 110, where it may exit shaft 108. The operator may then utilize imaging device 112 to help advance cytology snare 600 to a position adjacent the stricture before transitioning cytology snare 600 from the collapsed configuration to the expanded configuration. In some embodiments, elevator 116 may be utilized to aid in the positioning of cytology snare 600.

Once adjacent the structure, cytology snare 600 may be transitioned from the collapsed configuration to the expanded configuration via actuation wire 610, as described above. The operator may then utilize snare wire 602 to scrape or cut tissue from the stricture. Once tissue within the stricture has been dislodged by snare wire 602, a suction feature of medical device 100 may be utilize to suck the dislodged tissue through working channel 140 and into a biopsy trap.

After the tissue sample has been collected, cytology snare 600 may be transitioned back to the collapsed configuration so it may be removed from medical device 100 via shaft 108, and through port 130.

While principles of this disclosure are described herein with reference to illustrative examples for particular applications, it should be understood that the disclosure is not limited thereto. For example, the disclosure refers to ERCP as an exemplary procedure, and the bile and pancreatic ducts as typical lumens for the systems and methods of the disclosure. The systems, devices, and methods of the present disclosure, however, may be used in any suitable medical procedure in any lumen or cavity within the body, for example, to remove any unwanted material from the body. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A medical device, comprising:
an expandable tool configured to extend from a distal end of the medical device, the expandable tool comprising:
a plurality of wires extending from a proximal end of the expandable tool to a distal end of the expandable tool,
a plurality of fins extending outwardly from each of the wires of the plurality of wires, and
a plurality of projections extending outwardly from each of the fins of the plurality of fins.

2. The medical device of claim 1, wherein the plurality of wires defines a plurality of legs, and wherein each of the legs of the plurality of legs branch out from a central trunk disposed at the proximal end of the expandable tool.

3. The medical device of claim 2, wherein the plurality of legs are joined together at the distal end of the expandable tool.

4. The medical device of claim 1, wherein the expandable tool is configured to be transitioned from a collapsed configuration to an expanded configuration.

5. The medical device of claim 4, wherein, in the collapsed configuration, each of the fins of the plurality of fins is configured to collapse inward toward the plurality of wires.

6. The medical device of claim 4, wherein, in the expanded configuration, each of the wires of the plurality of wires is configured to bow outward such that each of the wires has a concave profile.

7. The medical device of claim 4, wherein, in the collapsed configuration, an outer diameter of the expandable tool is no greater than 1.5 mm.

8. The medical device of claim 4, wherein, in the expanded configuration, a width of the expandable tool is in a range of 1 mm to 15 mm.

9. The medical device of claim 1, wherein the wires of the plurality of wires comprise a first material, and wherein the fins of the plurality of fins comprise a second material.

10. The medical device of claim 9, wherein the first material is the same as the second material.

11. The medical device of claim 9, wherein the projections of the plurality of projections comprise a third material.

12. The medical device of claim 11, wherein the third material is the same as the second material.

13. The medical device of claim 1, wherein the fins of the plurality of fins are integrally formed with the wires of the plurality of wires.

14. The medical device of claim 1, wherein the projections of the plurality of projections extend radially outward relative to a longitudinal axis of each of the fins of the plurality of fins.

15. The medical device of claim 1, wherein the wires of the plurality of wires extend approximately parallel to a longitudinal axis of the expandable tool in a collapsed configuration of the expandable tool.

16. A medical device, comprising:
an expandable tool configured to extend from a distal end of the medical device, the expandable tool comprising:
a plurality of wires extending from a proximal end of the expandable tool to a distal end of the expandable tool,
wherein, in an expanded configuration of the expandable tool, each of the wires of the plurality of wires is configured to be curved outward to form a basket,
wherein each of the wires of the plurality of wires includes a first portion configured to extend inwardly relative to a profile of the wire, and a second portion configured to extend outwardly relative to the profile of the wire, and
wherein the wires of the plurality of wires are configured to be joined together at the distal end of the expandable tool.

17. The medical device of claim 16, wherein the wires of the plurality of wires include sharpened wires.

18. The medical device of claim 16, wherein the wires of the plurality of wires include textured wires.

19. A method for collecting a tissue sample using a medical device, the method comprising:
navigating an expandable tool to a target site within a lumen of a patient, the expandable tool comprising:
a plurality of wires extending from a proximal end of the expandable tool to a distal end of the expandable tool,
a plurality of fins extending outwardly from each of the wires of the plurality of wires, and
a plurality of projections extending outwardly from each of the fins of the plurality of fins;
transitioning the expandable tool from a collapsed configuration to an expanded configuration; and
contacting a wall of the lumen with the plurality of projections such that the tissue sample is collected on the projections.

20. The method of claim 19, further comprising, after contacting the wall of the lumen, transitioning the expandable tool from the expanded configuration to the collapsed configuration.
